(19) United States
(12) Patent Application Publication
(10) Pub. No.: US 2026/0309691 A1
(43) Pub. Date: Oct. 8, 2026

(54) VERFAHREN ZUM ÜBERPRÜFEN DES AUSHÄRTEVORGANGS VON BETON

(71) Applicant: Kraft Curing Systems GmbH, Lindern (DE)

(72) Inventor: Philipp Ennulat, Löningen (DE)

(21) Appl. No.: 19/529,704

(22) Filed: Feb. 4, 2026

(30) Foreign Application Priority Data
Apr. 2, 2025 (DE) 10 2025 103 975.5

(51) Int. Cl. G01N 33/38

(52) U.S. Cl. CPC G01N 33/383

(57) ABSTRACT

A method for testing the curing process of concrete and/or of concrete of concrete products, includes: preparing several test bodies of the concrete which is to be tested, with a defined mixture formula; carrying out pressure tests after established dwell times on the test bodies and inputting the pressure values obtained in these pressure tests into a data software; with the data software a pressure-time diagram is created, and this diagram is stored for the measured concrete formula in the data software; strength and equivalent time for the curing of the concrete are determined by means of the diagram which was created in the data software.

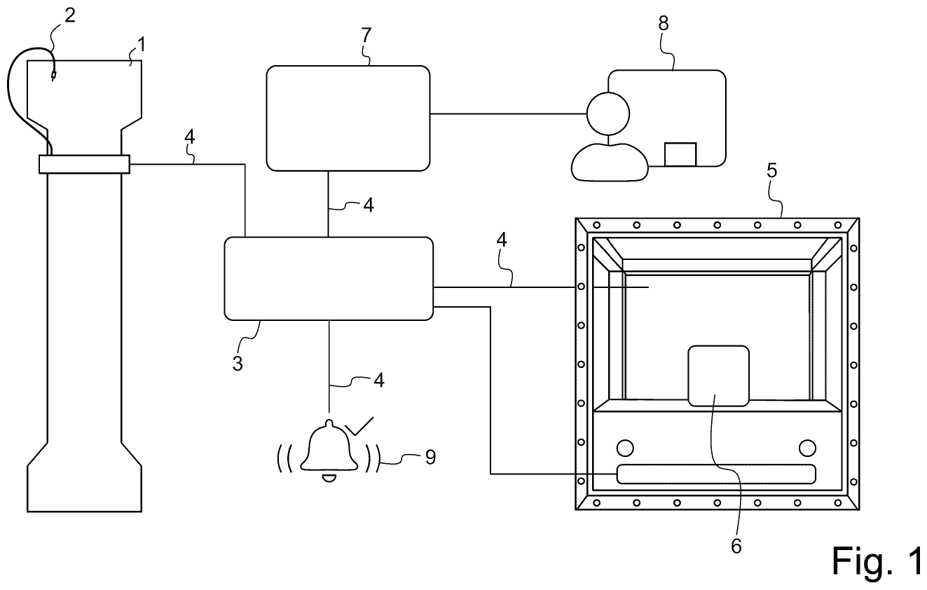

### FIELD OF THE INVENTION

The invention relates to a method for testing the curing process of concrete and/or of concrete of concrete products.

### BACKGROUND OF THE INVENTION

Several products are produced from concrete, particularly for structures. In particular, bridge pillars or also bridge portions are prefabricated from concrete and are then installed in a construction site in situ.

The produced concrete products are naturally to be manufactured so that they have a long service life. The fact that several bridges in Germany are dilapidated is currently the subject of public discussion. In this respect, the concrete of concrete products must harden completely before the concrete product is installed.

Added to this is that there are very many types of concrete which are different from one another, with different mixture formulae from one another for the concrete. Users of the different concrete types with the different mixture formulae are faced with the problem that in the case of each of these concretes they must test the curing process and must test the development up to final strength of the concrete.

### SUMMARY OF THE INVENTION

The invention is based on the problem of indicating a method of the type named in the introduction, by which the testing of the curing process of concrete is simplified, and by which in particular final strength developments of a concrete or of a concrete product can be predicted.

This problem is solved according to the invention by a method with the following steps:
preparing several test bodies from the concrete which is to be tested, with a defined mixture formula
carrying out pressure tests after established dwell times on the test bodies and inputting the pressure values obtained in these pressure tests into a data software
a pressure-time diagram is created with the data software, and this diagram is stored in the data software for the measured concrete formula
strength and equivalent time for the curing of the concrete are determined by means of the diagram created in the data software.

After running through this method according to the invention, the strength of a concrete or of the concrete of a concrete product can be calculated by means of the temperature-time relationship, the activation energy of the cement which is used and the created diagram, and can thus be predicted for temperature profiles which are different from one another. The data software helps here only to produce the diagram from pressure and time. The mixture formula is previously known, thus also the activation energy of the cement which is respectively used in the mixture formula.

In order to have a sufficient number of samples available, preferably approximately 20 test bodies are prepared. The mixture formula which is used is preferably a cement type with aggregates; for this actual mixture formula, by the method according to the invention a record of the relationship of temperature and time and of the activation energy of the cement which is used and of the created pressure-time diagram is calculated, in order to thus predetermine for the actual mixture formula the strength of a concrete product of this mixture formula for different temperature profiles.

The test bodies can be stored at approximately 20° C.

The method according to the invention can be tested. For this, provision is made that for a quality verification of this method during an after-treatment of the test bodies, test material of the same mixture formula is placed into a test oven and after-treated at the same temperature and is subjected to a pressure test.

With such a concluding pressure test, the achieved strength of the concrete product can be confirmed and documented.

### BRIEF DESCRIPTION OF THE DRAWINGS

An example embodiment of the method according to the invention is illustrated in the drawing. The single FIGURE of the drawing shows schematically the sequence of the method according to the invention for testing the curing process.

### DETAILED DESCRIPTION

The FIGURE shows a concrete product 1, which is for example part of a bridge structure. A sensor 2, which is linked with a control device 3 via a data line 4, is applied to the concrete product 1.

A test body 6 is arranged in a test oven 5. The test body 6 is formed from the same concrete as the concrete product 1 with the same defined mixture formula for the concrete.

Pressure tests can be carried out on the test body 6 in the test oven 5; the pressure values are delivered via data lines 4 and via the control device 3 to a data software 7. With the data software 7 a pressure-time diagram is created, and this diagram is stored in the data software 7 for the measured concrete formula. The data software 7 is monitored here at a monitor station 8.

If abnormal values occur in particular in the pressure tests, an alarm 9 can be triggered by the control device 3 via a data forwarding 4.

1. A method for testing the curing process of concrete and/or of concrete of concrete products, comprising:
preparing several test bodies of the concrete which is to be tested, with a defined mixture formula;
carrying out pressure tests after established dwell times on the test bodies and inputting the pressure values obtained in these pressure tests into a data software;
creating, with the data software a pressure-time diagram, and this diagram is stored in the data software (7) for the measured concrete formula; and
determining strength and equivalent time for the curing of the concrete by means of the diagram which is created in the data software (7).

2. The method according to claim 1, wherein approximately 20 test bodies are prepared.

3. The method according to claim 1, wherein a cement type with aggregates is used for the mixture formula of the test bodies.

4. The method according to claim 1, wherein for a quality verification of the method during an after-treatment of the test bodies, test material of the same mixture formula is placed in a test oven and is after-treated with the same temperature and is subjected to a pressure test.
